(19) United States
(12) Patent Application Publication
(10) Pub. No.: US 2026/0276618 A1
(43) Pub. Date: Sep. 17, 2026

(54) SYSTEMS AND METHODS FOR AUTOMATICALLY IDENTIFYING ANALOG MATERIALS BASED ON ESTIMATED PETROPHYSICAL PROPERTIES OF A SELECTED SAMPLE MATERIAL

(71) Applicant: BP Corporation North America Inc., Houston, TX (US)

(72) Inventors: Glen Gettemy, Katy, TX (US); Jennie Cook, Houston, TX (US); Nathan Lane, Katy, TX (US)

(73) Assignee: BP Corporation North America Inc., Houston, TX (US)

(21) Appl. No.: 19/553,758

(22) Filed: Mar. 2, 2026

Related U.S. Application Data
(60) Provisional application No. 63/773,216, filed on Mar. 17, 2025.

(51) Int. Cl. G01N 33/24; E21B 47/002

(52) U.S. Cl. CPC G01N 33/24; E21B 47/002

(57) ABSTRACT

A method for automatically identifying analog materials of a subject porous media sample includes receiving a set of digitally estimated petrophysical properties of a plurality of reference porous media samples from a material library, determining a similarity score between a subject porous media sample and each of the plurality of reference porous media samples by comparing a plurality of digitally estimated petrophysical properties of the subject porous media sample with the digitally estimated petrophysical properties of each of the plurality of reference porous media samples, and identifying one or more of the plurality of reference porous media samples as an analog material of the subject porous media sample in response to the similarity scores of the one or more reference porous media samples meeting a predefined similarity score threshold.

### CROSS-REFERENCE TO RELATED APPLICATIONS

This application claims benefit of U.S. provisional patent application Ser. No. 63/773,216 filed Mar. 17, 2025, and entitled “Systems and Methods for Automatically Identifying Analog Materials Based on Estimated Petrophysical Properties of a Selected Sample Material,” which is hereby incorporated herein by reference in its entirety for all purposes.

### STATEMENT REGARDING FEDERALLY SPONSORED RESEARCH OR DEVELOPMENT

Not applicable.

### BACKGROUND

In various applications, it may be desired to understand how fluid flows through and/or within a porous media. Such estimates may be dependent (at least partially) on the specific properties of the porous media and the fluid(s) flowing therein. As an example, in hydrocarbon exploration and production, the porous media in question may comprise a subsurface region (e.g., an Earthen subsurface region or formation). Obtaining accurate estimates of petrophysical properties of a subsurface region may thus be important for characterizing the concentration of fluids and/or minerals within the formation. Traditionally, samples are obtained from the formation (e.g., in the form of core samples or drilling cuttings) and subjected to laboratory testing whereby petrophysical properties such as permeability, porosity, formation factor, elastic moduli, and the like of the sample may be determined. Once determined, these properties can then be used to form predictions regarding the behavior of the subsurface region which may inform a strategy for configuring well systems which may be utilized for a variety of purposes including, for example, extracting hydrocarbons or other resources from the formation, carbon capture and storage within the formation, and other applications. As an example, the determined properties may be utilized in determining the location of a planned wellbore for producing hydrocarbons from the subsurface region and/or the configuration of the planned wellbore.

### BRIEF SUMMARY OF THE DISCLOSURE

An embodiment of a computer implemented method for automatically identifying analog materials of a subject porous media sample comprises (a) receiving a set of digitally estimated petrophysical properties of a plurality of reference porous media samples from a material library, (b) determining a similarity score between a subject porous media sample and each of the plurality of reference porous media samples by comparing a plurality of digitally estimated petrophysical properties of the subject porous media sample with the digitally estimated petrophysical properties of each of the plurality of reference porous media samples, and (c) identifying one or more of the plurality of reference porous media samples as an analog material of the subject porous media sample in response to the similarity scores of the one or more reference porous media samples meeting a predefined similarity score threshold. In some embodiments, (b) comprises (b1) statistically transforming each of the digitally estimated petrophysical properties of the plurality of reference porous media samples to generate a corresponding plurality of transformed petrophysical properties of the plurality of reference porous media samples, (b2) statistically transforming each of the digitally estimates petrophysical properties of the subject porous media sample to generate a corresponding plurality of transformed petrophysical properties of the reference porous media sample, and (b3) determining the similarity score by comparing the plurality of transformed petrophysical properties of the subject porous media sample with the plurality of transformed petrophysical properties of each of the plurality of reference porous media samples. In certain embodiments, (b1) comprises applying a Z-score statistical transformation to each of the digitally estimated petrophysical properties of the plurality of reference porous media samples, and (b2) comprises applying a Z-score statistical transformation to each of the digitally estimates petrophysical properties of the subject porous media sample. In other embodiments, the similarity score comprises a set of Euclidean distances between the plurality of transformed petrophysical properties of the subject porous media sample and the plurality of transformed petrophysical properties of each of the plurality of reference porous media samples. In some embodiments, (b3) comprises individually weighting one or more of the plurality of transformed petrophysical properties of the subject porous media sample and the plurality of transformed petrophysical properties of each of the plurality of reference porous media samples. In certain embodiments, (b) comprises defining a set of user-selectable weights for each of the digitally estimated petrophysical properties of the plurality of reference porous media samples. In other embodiments, (b) further comprises incorporating the set of the user-selectable weights in the similarity score. In some embodiments, (b) comprises ranking the plurality of reference porous media samples in order of decreasing similarity score.

An embodiment of a computer-implemented method for automatically identifying analog materials of a subject porous media sample comprises (a) receiving a set of digitally estimated petrophysical properties of a plurality of reference porous media samples from a material library, (b) determining a similarity score between a subject porous media sample and each of the plurality of reference porous media samples by comparing a plurality of digitally estimated petrophysical properties of the subject porous media sample with the digitally estimated petrophysical properties of each of the plurality of reference porous media samples, (c) adjusting the similarity score based on an individual weighting of each of the digitally estimated petrophysical properties of the subject porous media sample and each of the plurality of reference porous media samples, and (d) identifying one or more of the plurality of reference porous media samples as an analog material of the subject porous media sample based on a ranking of each of the digitally estimated petrophysical properties of the plurality of reference porous media samples and the similarity score. In some embodiments, (b) comprises (b1) statistically transforming each of the digitally estimated petrophysical properties of the plurality of reference porous media samples to generate a corresponding plurality of transformed petrophysical properties of the plurality of reference porous media samples, (b2) statistically transforming each of the digitally estimates petrophysical properties of the subject porous media sample to generate a corresponding plurality of transformed petrophysical properties of the reference porous media sample, and (b3) determining the similarity score by comparing the plurality of transformed petrophysical properties of the subject porous media sample with the plurality of transformed petrophysical properties of each of the plurality of reference porous media samples. In certain embodiments, (b1) comprises applying a Z-score statistical transformation to each of the digitally estimated petrophysical properties of the plurality of reference porous media samples. In other embodiments, (b2) comprises applying a Z-score statistical transformation to each of the digitally estimates petrophysical properties of the subject porous media sample. In some embodiments, (c) comprises individually weighting the digitally estimated petrophysical properties making up the plurality of digitally estimated petrophysical properties of the subject porous media sample and the plurality of digitally estimated petrophysical properties of each of the plurality of reference porous media samples. In certain embodiments, (c) further comprises defining a set of user-selectable weights for each of the digitally estimated petrophysical properties of the plurality of reference porous media samples. In other embodiments, (c) further comprises incorporating the set of the user-selectable weights in the similarity score. In some embodiments, (d) comprises ranking the plurality of reference porous media samples in order of decreasing similarity score.

An embodiment of a system for automatically identifying analog materials of a subject porous media sample comprises a one or more processors, and a storage device coupled to the one or more processors, the storage device configured to store instructions that, when executed by the one or more processors, configure the one or more processors to (a) receive a set of digitally estimated petrophysical properties of a plurality of reference porous media samples from a material library, (b) determine a similarity score between a subject porous media sample and each of the plurality of reference porous media samples by comparing a plurality of digitally estimated petrophysical properties of the subject porous media sample with the digitally estimated petrophysical properties of each of the plurality of reference porous media samples, and (c) identify one or more of the plurality of reference porous media samples as an analog material of the subject porous media sample in response to the similarity scores of the one or more reference porous media samples meeting a predefined similarity score threshold. In some embodiments, the storage device is configured to store instructions that, when executed by the one or more processors, further configure the one or more processors to (b1) statistically transform each of the digitally estimated petrophysical properties of the plurality of reference porous media samples to generate a corresponding plurality of transformed petrophysical properties of the plurality of reference porous media samples, (b2) statistically transform each of the digitally estimates petrophysical properties of the subject porous media sample to generate a corresponding plurality of transformed petrophysical properties of the reference porous media sample, and (b3) determine the similarity score by comparing the plurality of transformed petrophysical properties of the subject porous media sample with the plurality of transformed petrophysical properties of each of the plurality of reference porous media samples. In certain embodiments, the storage device is configured to store instructions that, when executed by the one or more processors, further configure the one or more processors to (b1) apply a Z-score statistical transformation to each of the digitally estimated petrophysical properties of the plurality of reference porous media sample, and (b2) apply a Z-score statistical transformation to each of the estimated petrophysical properties of the subject porous media sample. In other embodiments, the storage device is configured to store instructions that, when executed by the one or more processors, further configure the one or more processors to (b3) individually weight one or more of the transformed petrophysical properties of the subject porous media sample and the plurality of transformed petrophysical properties of each of the plurality of reference porous media samples.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a block diagram of a computer-implemented method for automatically identifying analog materials based on estimated petrophysical properties of a selected sample material in accordance with principles disclosed herein;

FIG. 2 is a schematic view of exemplary onshore and offshore sources of acquiring and analyzing core samples for purposes of generating digital image volumes of core samples and fluid sampling for analysis by embodiments of testing systems and methods in accordance with principles disclosed herein;

FIG. 3 is a schematic view of an embodiment of a testing system for analyzing core samples in accordance with principles disclosed herein;

FIG. 4 is an exemplary digital image volume after being segmented into a plurality of separate volume elements in accordance with principles disclosed herein;

FIG. 5 is an embodiment of an exemplary material library for storing estimated petrophysical properties of a plurality of reference porous media in accordance with principles disclosed herein;

FIGS. 6 and 7 are graphs illustrating exemplary petrophysical property distributions in accordance with principles disclosed herein;

FIG. 8 is a graph illustrating similarity scores of porous media samples in accordance with principles disclosed herein;

FIG. 9 is an embodiment of a system for implementing method 10 of FIG. 1 in accordance with principles disclosed herein;

FIG. 10 is an embodiment of an exemplary method for developing a material library in accordance with principles disclosed herein;

FIG. 11 is a block diagram of a computer-implemented method for automatically identifying analog materials of a subject porous media sample in accordance with principles disclosed herein;

FIG. 12 is a block diagram of another computer-implemented method for automatically identifying analog materials of a subject porous media sample in accordance with principles disclosed herein; and

FIG. 13 is a block diagram of an exemplary computer system suitable for implementing one or more embodiments in accordance with principles disclosed herein is shown.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis.

As previously described, an accurate understanding of material flow (e.g., fluid flow) within a porous media may be dependent upon specific parameters and characteristics of the porous media and materials contained therein and forming the porous media. When the porous media comprises a subsurface region (e.g., such as in the context of hydrocarbon exploration and production), the properties and behavior of the subsurface region may be directly measured via samples obtained from the formation itself (e.g., in the form of core samples, drill cuttings, and the like). However, due to the cost and time required to directly measure petrophysical properties of formation samples of porous media (also referred to herein as “core samples” or “rock samples”), the technique of “direct numerical simulation” can be applied to efficiently predict or estimate physical properties of the subsurface region, such as porosity, absolute permeability, relative permeability, formation factor, elastic moduli, and the like of core samples, including samples from difficult rock types such as tight gas sands or carbonates.

According to this approach, a three-dimensional (3D) tomographic image volume of the core sample is obtained, for example, by way of a computer tomographic (CT) scan. The 3D image volume is segmented into a plurality of distinct volume elements or “voxels” (e.g., by “thresholding” their brightness values or by another approach) to distinguish rock matrix from void space in the core sample. Direct numerical simulation of fluid flow or other physical behavior such as elasticity or electrical conductivity is then performed, from which porosity, permeability, elastic properties, electrical properties, and the like can be derived. A variety of numerical methods may be applied to solve or approximate the physical equations simulating the appropriate behavior of the core sample. These methods include, for example, lattice-Boltzmann (LB), finite element, finite difference, finite volume, numerical methods and the like.

Ultimately, as previously described, it is desirable to model the movement of fluid within a subsurface region. Accurate modeling of such behavior may be relevant for hydrocarbon production operations (e.g., determining well placement and/or configuration), and also for other considerations such as contaminant transport and rock diagenesis. The movement of fluid within a subsurface region is a function of fluid properties, and the various petrophysical properties of the rock (e.g., permeability, porosity, formation factor, elastic moduli, etc.), which may be determined as generally described above. In addition, identifying analog samples based on estimated petrophysical properties of a sample material (e.g., rock sample) is crucial in obtaining insights into fluid movement within a subsurface region. For example, by comparing the petrophysical properties of a sample material with those of previously stored samples, scientists can identify analogs and thus, predict subsurface characteristics, such as fluid flow and storage capacity, without direct access to the subsurface region being evaluated. Moreover, identifying suitable analogs allow data from well understood subsurface regions to be incorporated into less understood ones. For instance, analogs obtained from material libraries/databases can serve as proxies for subsurface regions by providing properties like lithology and fluid distribution patterns, which may reduce the uncertainties in geological models and enhance the reliability of predictions regarding hydrocarbon well performance. However, careful selection of appropriate analogs is crucial, as discrepancies in petrophysical properties may result in inaccurate models and unreliable forecasts.

Accordingly, embodiments disclosed herein includes computer implemented systems and methods for automatically identifying analog materials based on digitally estimated petrophysical properties of a subject porous media sample. Particularly, embodiments disclosed herein utilize digital images of rock samples along with digitally estimated petrophysical properties of a plurality of reference porous media samples from a material library to identify analogs of the subject porous media sample. Embodiments disclosed herein further includes determining a similarity score between a subject porous media sample and each of the plurality of reference porous media samples, and identifying one or more of the plurality of reference porous media samples as an analog material of the subject porous media sample in response to the similarity scores of the one or more reference porous media samples meeting a predefined similarity score threshold. In some embodiments, the porous media sample comprises a subterranean formation. Thus, through use of the embodiments disclosed herein, more accurate identification of suitable analogs may be made for porous media (e.g., a subterranean formation), which may further enhance various operations, such as hydrocarbon extraction, environmental mitigation, etc.

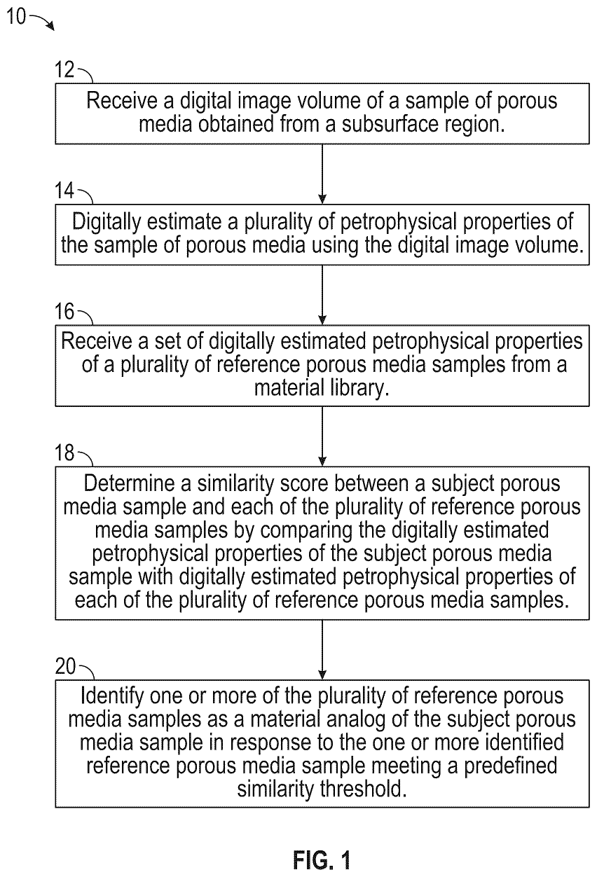

Referring to FIG. 1, an embodiment of a computer-implemented method 10 for automatically identifying analog materials of a subject porous media sample based on digitally estimated petrophysical properties of the subject porous media sample is shown. As used herein, the term “digitally estimated” refers to petrophysical properties of a porous media sample that are estimated based on a digital image volume of the porous media sample. In some embodiments, the subject porous media sample comprises a core or other rock sample obtained from a subsurface region.

Initially, at block 12, method 10 comprises receiving a digital image volume of a subject sample of porous media (e.g., a core sample and the like) obtained from a subsurface region. The digital image volume is a three-dimensional (3D) representation of the porous media sample (e.g., a core or other sample obtained from a subsurface region). The subject porous media sample may be initially digitally imaged with the obtained image comprising a digital image. In some embodiments, the digital image volume received at block 12 may comprise a 3D tomographic image of the subject porous media sample. For instance, the 3D tomographic image may be obtained by a computer tomographic (CT) scan of the subject porous media sample. As will be discussed further herein, the 3D image may be segmented into a plurality of distinct volume elements or voxels to distinguish rock matrix of the core sample from void or pore space thereof.

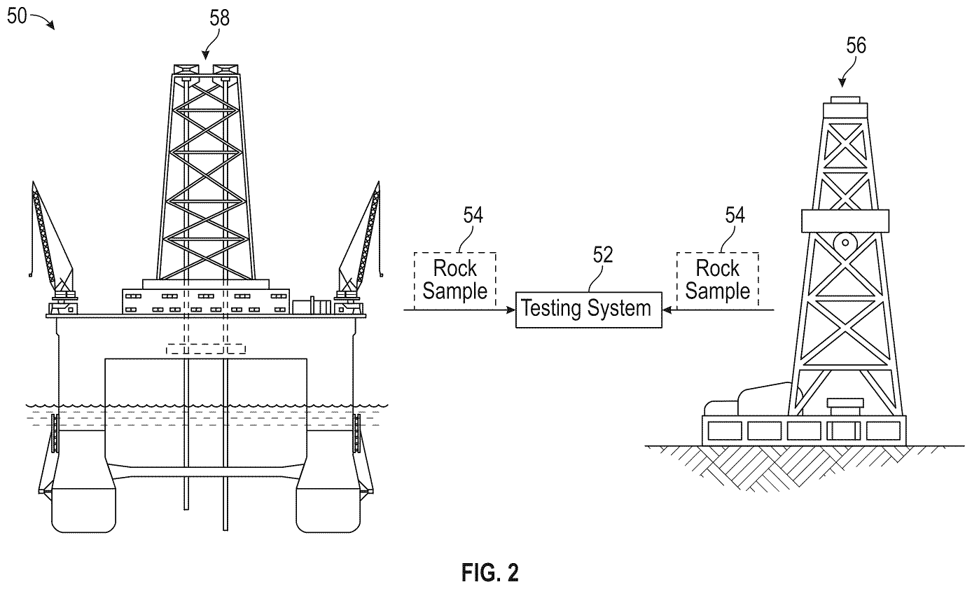

To provide an example, and referring to FIG. 2, a system 50 for acquiring and analyzing core samples 54 for purposes of generating digital image volumes of samples of porous media (e.g., the digital image volume received in block 12 of method 10 in FIG. 1) is shown in FIG. 2 according to an exemplary embodiment. In some embodiments, system 50 may be used to analyze samples of porous media in the form of core samples 54 that are obtained from a subsurface region for purposes of enhancing or facilitating oil and gas production from the subsurface region. For example, in some embodiments core samples 54 can be obtained from terrestrial drilling system 56 or from marine (ocean, sea, lake, etc.) drilling system 58, either of which is utilized to extract resources such as hydrocarbons (oil, natural gas, etc.), water, and the like.

The way core samples 54 are obtained and the physical form of those samples can vary widely. Examples of core samples 54 useful in connection with embodiments disclosed herein include whole core samples, sidewall core samples (e.g., obtained from the sidewall of a wellbore extending through the subsurface region), outcrop samples, drill cuttings, and laboratory generated synthetic core samples such as sand packs and cemented packs.

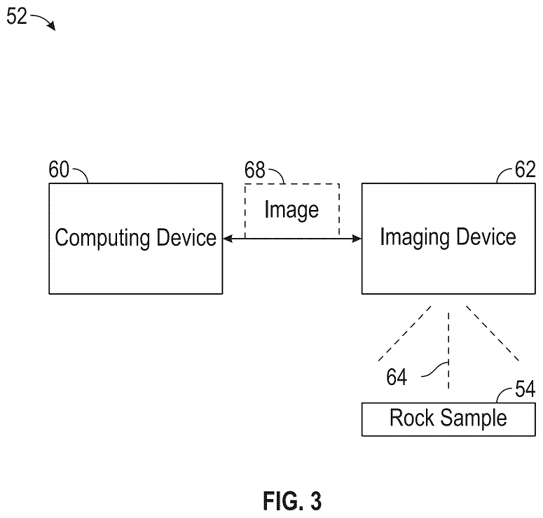

A testing system 52 of system 50 is configured to acquire and analyze (3D) digital image volumes 68 (shown in FIG. 3) of core samples 54 to determine the physical properties of the corresponding sub-surface rock. The physical properties include, for example, petrophysical properties such as those petrophysical properties that are analyzed in the context of oil and gas exploration and production.

Referring to FIG. 3, a schematic diagram of testing system 52 is shown according to some embodiments. Testing system 52 includes imaging device 62 for obtaining 2D or 3D digital image volumes, as well as other representations, of core samples 54, such digital image volumes and representations including details of the internal structure of the core samples 54. In some embodiments, imaging device 62 may be used to obtain 2D or 3D digital image volumes of a core sample 54 either when the core sample 54 is received in an at least partially surrounding sample receptacle or when the core sample 54 is free of any external receptacle or structure.

An example of imaging device 62 is an X-ray CT scanner (or more simply “CT scanner”), which emits X-ray radiation 64 that interacts with an object and measures the attenuation of that X-ray radiation 64 by the object to generate a digital image volume of its interior structure and constituents. The particular type, construction, or other attributes of CT scanner 62 can correspond to that of any type of X-ray device, such as a micro-CT scanner, capable of producing a digital image volume representative of the internal structure of core sample 54. Other embodiments of imaging device 62 may utilize other imaging techniques, such as, X-ray nano-tomography, focused ion beam scanning electron microscopy, nuclear magnetic resonance, etc.

In some embodiments, the digital image volume may be computationally generated rather than produced by scanning the core samples 54. In embodiments in which the digital image volume 68 is produced by scanning a core sample 54, the core sample 54 may be a naturally occurring rock or a man-made porous material (e.g., a synthetic rock or other porous media).

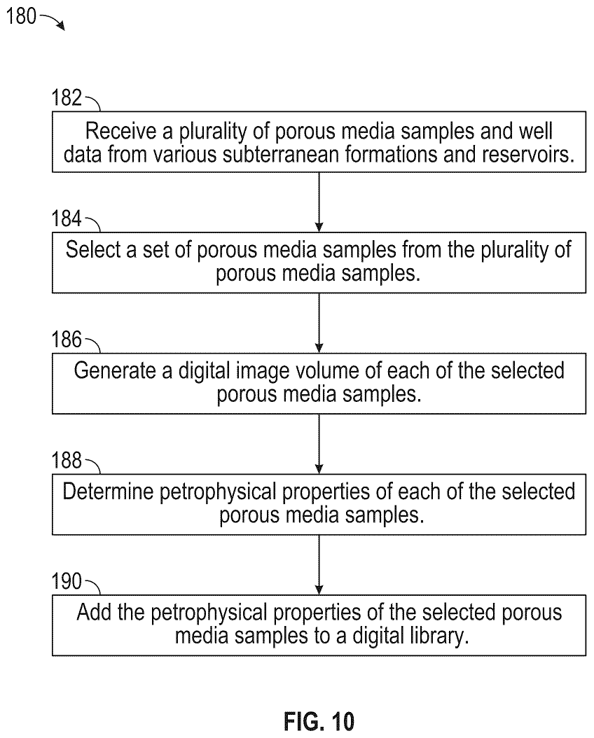

Digital image volume 68 may be composed of grayscale values representative of the attenuation of the X-ray radiation by the constituents of core sample 54. In addition, digital image volume 68 may comprise multiple two-dimensional (2D) slice images stacked along an axis of the core sample 54, which together form the digital image volume 68 of core sample 54. In general, the stacking of the 2D slice images into digital image volume 68 may be performed by computational resources of imaging device 62 itself, or by a separate computing device 60 from the series of 2D slice images produced by imaging device 62, depending on the particular architecture of testing system 52. The computing device 60 may comprise or be similar to the computer system 400 shown in FIG. 10.

The digital image volumes produced by imaging device 62 may be partitioned into 3D regular elements called volume elements, or more commonly “voxels”. For example, each voxel may be cubic, having a side of equal length in the orthogonal X, Y, and Z directions. In some embodiments, the digital image volumes produced by the imaging device 62 may be partitioned into non-cubic grid volume elements having varying geometries. The digital image volume 68 itself may contain different numbers of voxels in the X, Y, and Z directions. Each voxel within a respective 3D digital image has an associated numeric value, or amplitude, that represents the relative material properties of the imaged sample at that location of the medium represented by the digital volume. The range of these numeric values (commonly known as the grayscale range) depends on the type of digital volume, the granularity of the values, the size of the data register (e.g., 8-bit or 16-bit values), and the like. For example, 16-bit data values enable the voxels of an X-ray tomographic digital image volume to have amplitudes ranging from 0 to 65,536 with a granularity of 1. In some embodiments, testing system 52 may perform image enhancement techniques (e.g., data smoothing, noise reduction) on the digital image volume. Likewise, other components of testing system 52 (e.g., imaging device 62 itself) may alternatively perform image enhancement in whole or in part. The digital image volumes 68 captured by the imaging device 62 may comprise the digital image volumes received at block 12 of method 10.

Referring again to FIG. 1, at block 14, one or more rock or petrophysical properties of the subject porous media sample (e.g., the core sample) are automatically and digitally estimated based on the digital image volume of the subject porous media sample. The petrophysical properties estimated at block 14 may include, among other parameters, transport properties (e.g., electrical resistance, absolute permeability, relative permeability), structural properties (e.g., bulk modulus, grain size, porosity, formation factor, elastic modulus), and morphological properties (e.g., surface area, representative elementary volume (REV)).

As an example, REV refers to the volumetric extent of a porous media sample from which computational experiments or physical measurements may return values that are representative of the larger, or macroscopic, homogeneous rock mass. That is, the REV is defined as the subvolume size at which the physical parameter being computed or measured from the subvolume is not dependent on the particular location of the subvolume within the overall mass. Conversely, the data from computational measurements or experiments made on a computational domain or core sample of a volume smaller than the REV may not accurately represent the pore system of the rock mass macroscopically, but the physical parameter being computed or measured will vary depending on the location of the computational domain within the rock mass. In some embodiments, the REV of the porous media sample may be estimated from the digital image volume by defining (e.g., by computing device 60 shown in FIG. 3) a set of subvolume sizes, each subvolume size corresponding to a unique number of voxels among the set of subvolume sizes. For each of the set of subvolume sizes, one or more pairs of adjacent portions of the digital image volume having that subvolume size may be analyzed. Additionally, one of the subvolume sizes of the set of subvolume sizes is selected for analysis. Further, a pair of subvolumes of a size equal to the selected subvolume size may be acquired from the digital image volume, and located adjacent to one another in the digital image volume.

In certain embodiments, one or more material or petrophysical properties for each of the selected adjacent subvolumes may be determined based on direct numerical simulation or other numerical or synthetic methods. Consistent with the above, these petrophysical properties comprise physical properties of the material of the core sample that is represented by the digital image volume including, for example, porosity, permeability, relative permeability, electrical properties, elastic properties, geometrical properties, nuclear magnetic resonance (NMR), and the like. For example, electrical properties that may be determined include such properties as formation factor, resistivity index, tortuosity factor, cementation exponent, and saturation exponent. Additionally, elastic properties that may be determined include such properties as bulk modulus, shear modulus, Young's modulus, Poisson's ratio, compressional wave velocity, and shear wave velocity. Further, other petrophysical properties that may be determined include correlation lengths, surface to volume ratio, tortuosity, chord lengths, pore throat radii, pore size, pore shape, grain size, and grain shape, and the like.

In an example, porosity can be obtained for a segmented derivative subvolume by dividing the total number of pore space voxels by the total number of voxels contained within the subvolume. As another example, absolute permeability may be determined based on a variety of numerical methods such as finite element, finite difference or LB methods. These numerical approaches may simulate the physics of fluid flow (e.g., single phase fluid flow) to determine permeability by either directly solving/approximating the Navier-Stokes equations or recovering the Navier-Stokes equation from a discretization of the LB equation. Additionally, geometrical petrophysical properties, such as correlation lengths, chord lengths, etc. can be obtained based on Monte Carlo-like methods, where certain characteristics are randomly sampled throughout each adjacent subvolume. For instance, the correlation length can be estimated by randomly sampling two points displaced at a given distance.

In some embodiments, a difference value between the petrophysical property values discussed above for the adjacent subvolumes of the digital image volume is determined. For example, this difference value may represent the percentage or fractional difference in the petrophysical property values between those two adjacent portions of the digital image volume, at the current subvolume size. In certain embodiments, it may be determined whether additional one or pairs of subvolumes are to be selected and analyzed. The decision of whether or not additional subvolumes are to be selected may be based on the value of a counter that determines whether a pre-selected number of subvolume pairs to be analyzed for the current subvolume size has been completed. The process may be repeated with the selection of another pair of adjacent subvolumes at the current subvolume size if it is determined that additional subvolumes are to be selected, followed by the determination of the petrophysical properties of these additional subvolumes in order to determine a difference value for the additional subvolumes.

Conversely, in some embodiments, if the desired number of subvolume pairs has been analyzed for the current subvolume size, the mean of the difference values obtained over for the set of adjacent subvolumes at the current subvolume size may be determined. One or more other statistics that reflect the variance of the petrophysical property values between adjacent pairs of subvolumes at this current subvolume size may alternatively or additionally be calculated from these results. The mean difference value (or such other statistics) over the set of pairs of adjacent subvolumes at this subvolume size may be used to determine a difference value that is representative of the current specific subvolume size.

In alternative embodiments, following each instance of determining the one or more petrophysical properties for each pair of adjacent subvolumes, a cumulative mean difference value at the current subvolume size for the subvolume pairs analyzed so far for the current subvolume size may be determined. The cumulative mean difference value for the current subvolume size may provide a measure of convergence that is useful in determining whether additional subvolumes at the current subvolume size are to be selected for analysis. Convergence may be based on whether the calculated cumulative mean difference value changed after the most recent instance of determining the cumulative mean different value at the subvolume size, or on some other measure or statistic derived from this cumulative mean difference value. Additionally, if convergence has not yet been reached, another pair of adjacent subvolumes is selected at the current subvolume size and the cumulative mean difference value is determined for the new pair of subvolumes.

Upon determining that no additional subvolume pairs are to be selected, it may be subsequently determined whether this exemplary process is to be repeated for additional subvolume sizes. For example, the process may be performed on a predefined set of subvolume sizes, in which case it may simply be determined whether the predefined set has been exhausted. Alternatively, the mean difference values for the subvolume sizes processed so far may be analyzed, for example by analyzing a plot or statistical representation of those mean difference values to determine whether a REV that meets a predefined difference value or variance has yet been identified. Likewise, a plot of the mean difference values can be utilized to determine the uncertainty in material properties that have been calculated or numerically simulated so far over portions of the digital image volume at different sizes.

In certain embodiments, if additional subvolume sizes are to be analyzed, the exemplary process described above is repeated to select the next additional subvolume size. The different subvolume sizes may be selected to determine the mean difference value over multiple different sized portions of the digital image volume. For example, different subvolume sizes may be incrementally selected to include either a greater number of voxels or fewer voxels.

Upon determining that no additional subvolume sizes remain to be analyzed, the REV for the core sample currently being analyzed may be determined. For example, by calculating the difference values and the REV, an optimum (in at least some application) size of the subvolume may be determined that minimizes the uncertainty in the material properties simulated due to heterogeneity within the input volume. Particularly, a size of the subvolume may be determined (corresponding to the size of the REV) that minimizes the uncertainty in the material property values without unduly increasing the size of a portion of the subvolume to analyze.

In addition to REV determination, petrophysical parameters of the porous material sample may also be determined based on the digital image volume. For example, porosity for the porous media sample can be obtained by dividing the total number of pore space voxels contained in the digital image volume by the total number of voxels contained within the digital image volume. As a further example, absolute permeability, relative permeability, along with other petrophysical properties of the porous media sample may be determined from the digital image volume based on a variety of numerical methods such as finite element, finite difference or LB methods which simulate the physics of fluid flow to determine the petrophysical properties of interest. Further, geometrical petrophysical properties of the porous media sample, such as correlation lengths, chord lengths, etc. can be obtained from the digital image volume using Monte Carlo-like methods.

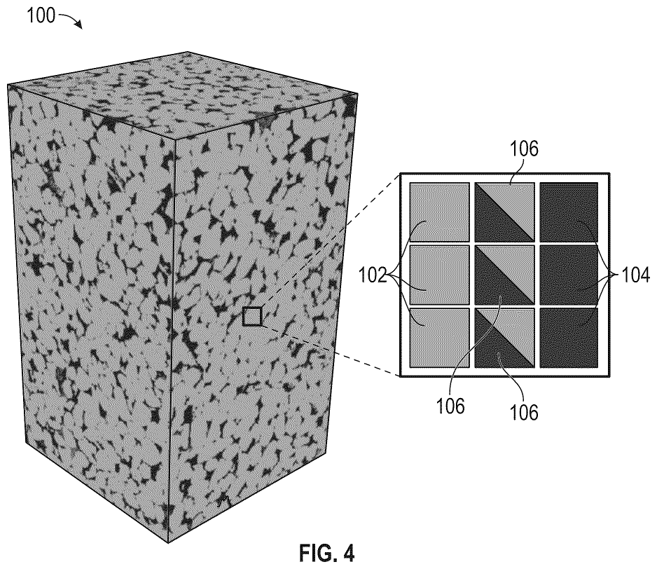

To provide another example, and referring briefly to FIG. 4, an exemplary digital image volume 100 is shown schematically as including a plurality of separate voxels. In some embodiments, the digital image volume 68 of FIG. 3 may be configured similarly or otherwise have features in common with digital image volume 100. Alternatively, the digital image volume 68 of FIG. 3 may vary in configuration from digital image volume 100 in other embodiments. Particularly, FIG. 4 illustrates digital image 100 as including a plurality of solid voxels 102 and a plurality of fluid voxels 104. In some embodiments, digital image volume 100 also includes one or more solid-fluid or interface voxels 106 that represent the overlap of the “solid-phase” and the “fluid-phase” where both solid and fluid properties may be tracked simultaneously. Alternatively, digital image volume 100 may only include solid voxels 102 and fluid voxels 104 but not interface voxels 106. The spatial configuration of the plurality of interface voxels 106 defines an interface of the digital image 100 between the solid-phase and fluid-phase constituents thereof.

Referring again to FIG. 1, in some embodiments, the petrophysical properties of the subject porous media sample digitally estimated at block 14 may be provided or added to a material library for storage therein. Particularly, the petrophysical properties may be indexed in the material library with a corresponding label or identifier of the given porous media sample. The identifier of the porous media sample may be based on the type of material forming the porous media sample, the location at which the porous media was sampled, or other identifying characteristics. In other embodiments, the identifier of the porous media sample may be arbitrarily or randomly assigned. In still other embodiments, the estimated petrophysical properties of the given porous media sample may not be added to the material library and instead remain separate from the material library.

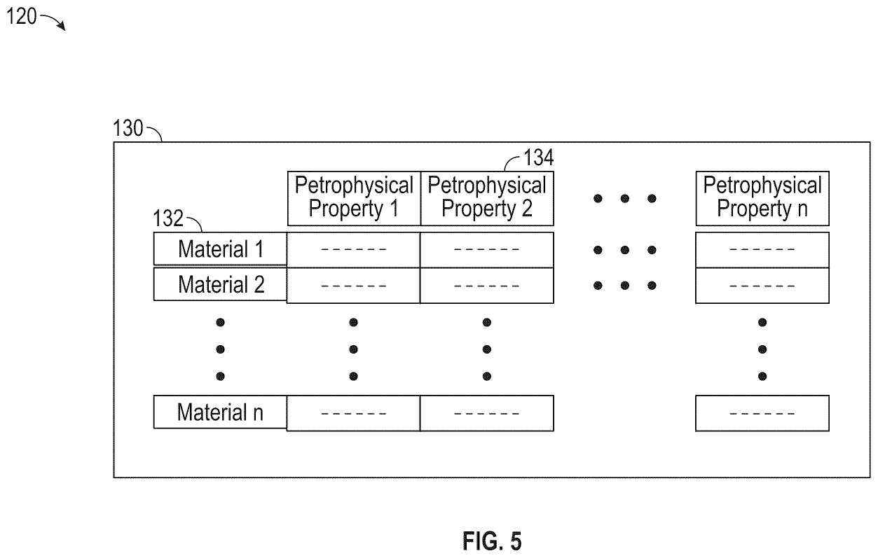

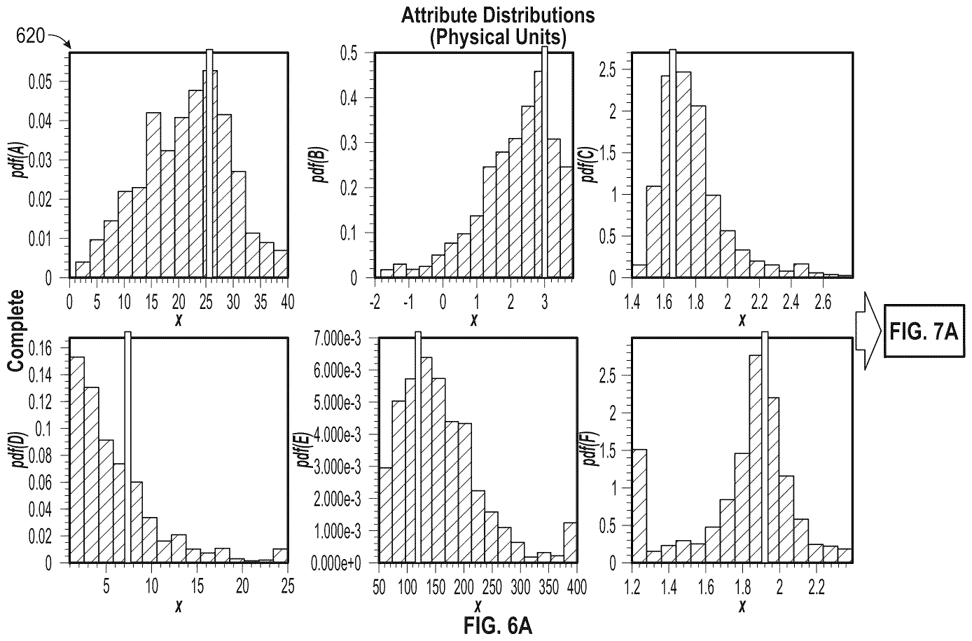

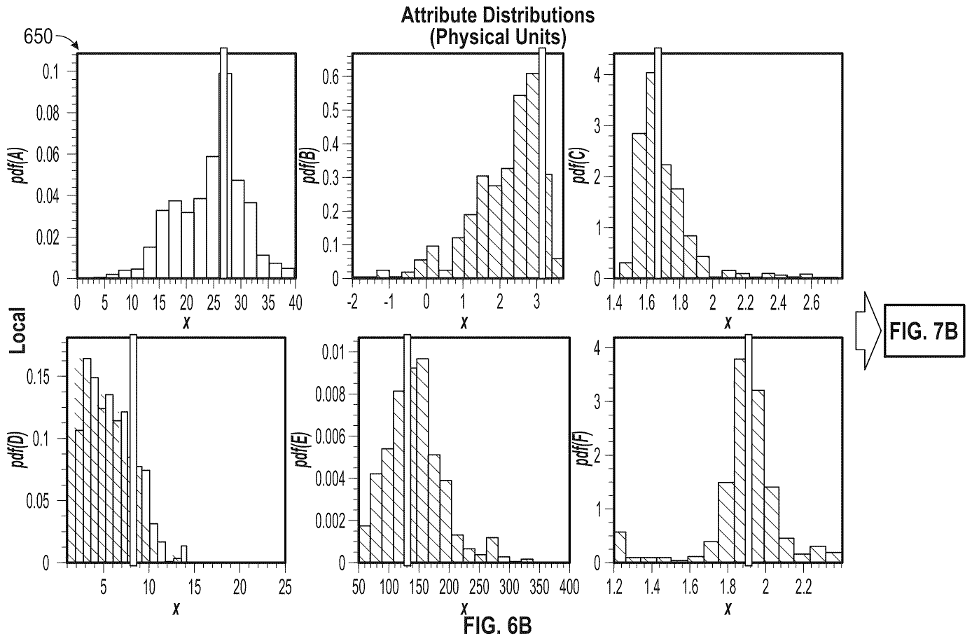

Referring to FIG. 5, an embodiment of a material library 120 is shown schematically. In this exemplary embodiment, material library 120 includes a digital database 130 comprising a collection of n distinct materials or porous media samples 132, each representing a type of porous media or core sample from a subterranean formation and associated with a corresponding n petrophysical property 134. Material library 120 may be contained in a datastore or other structure of a computer system. In this exemplary embodiment, database 130 catalogs the petrophysical properties 1 through n associated with each of the n porous media samples 132. For example, each of the n porous media samples 132 may be categorized based on its composition (e.g., material 1=sandstone, material 2=shale, material 3=limestone, material 4=clay, material n=synthetic material and so on) or other defining features. In another example, each of the n porous media samples 132 may be categorized based on the location in which the material was obtained within a particular subterranean formation. As previously described, the petrophysical properties may comprise physical properties of the material including, for example, porosity, permeability, relative permeability, electrical properties, elastic properties, geometrical properties, NMR, and the like. In some embodiments, material library 120 may also include metadata, for example, the date, source or well from which the material was obtained to provide context for users. Material library 120 may be created and continually updated as blocks 12 and 14 of the method 10 shown in FIG. 1 are performed for new material samples.

Returning to FIG. 1, at block 16, method 10 includes receiving a set of digitally estimated petrophysical properties of a plurality of different reference porous media samples from the material library. The porous media samples of the material library may be obtained via a variety of means including, for example, core sampling. Not intending to be bound by any particular theory, the set of porous media samples may be represented by the set “Sₖ” where “k” represents the number of different porous media samples captured in the set Sₖ, where k is less than or equal to “N”, which represents the total number of different porous media samples contained in the material library. In other words, the set of porous media samples corresponding to the set of digitally estimated petrophysical properties may be equal to or less than the total number of porous media samples contained in the material library. Thus, the set of porous media samples may itself comprise a sample of the total number of porous media samples referenced in the media library. As an example, the set of petrophysical properties received at block 16 may comprise the set of petrophysical properties 134 of the material library 120 shown in FIG. 5 for either the entirety or a subset of the different porous media samples 132 contained in the material library 120. Additionally, each member of the set Sₖ may include a corresponding set M of different digitally estimated petrophysical properties.

The digitally estimated petrophysical properties for each porous media sample stored in the material library (e.g., material library 120 shown in FIG. 5) may be determined based on the digital image volume of the porous media sample and applying, for example, numerical methods to estimate the petrophysical properties of each material as described further above with respect to block 14 of method 10.

The digitally estimated petrophysical properties of the materials in the material library may be indexed with a corresponding label or identifier of the given porous media sample and stored as reference porous media samples. In this manner, data on various properties or characteristics including petrophysical properties that have been digitally estimated for the reference porous media samples may be accessed and retrieved by (manually by a user or automatically) querying the material library. For example, when a subject porous media sample (i.e., material to be analyzed) has been characterized by a 3D image based on pore scale simulation, to characterize transport properties, structural properties and/or morphological properties of the porous media sample, the attributes associated with the subject 3D image of the porous media sample can be compared against equivalent attributes of reference porous media samples stored in the material library by querying the material library. For instance, the query may be based on specific petrophysical properties, porous media sample type, or similarity scores as will be disclosed further herein, to retrieve a set of digitally estimated petrophysical properties of a plurality of different reference porous media samples.

At block 18, method 10 includes determining a similarity score between the subject porous media sample and each of the plurality of different reference porous media samples by comparing the digitally estimated petrophysical properties of the subject porous media sample with digitally estimated petrophysical properties of each of the plurality of reference porous media samples. In some embodiments, determining a similarity score between the subject porous media sample and each of the plurality of reference porous media samples include using numerical (e.g., statistical) methods to quantitatively determine the similarity of the attributes (e.g., petrophysical properties) of the subject porous media sample with respect to the attributes of the reference porous media samples received from the material library. In certain embodiments, block 18 includes applying a predefined statistical transform to the digitally estimated set of petrophysical properties received at block 16 to generate a set of transformed petrophysical properties which standardize the set of petrophysical properties in accordance with the selected statistical transform. As used herein, the term “statistical transform” is defined as meaning a mathematical operation applied to a dataset (e.g., the set of digitally estimated petrophysical properties of sampled porous media samples obtained from a material library) to adjust its distribution or scale, such as for the purpose of normalization and/or standardization.

For example, block 18 may include applying a Z-score statistical transform to the digitally estimated set of petrophysical properties received at block 16, transforming the digitally estimated set of petrophysical properties into a Student's t-statistic to generate a set of transformed petrophysical properties that are standardized according to the Z-score transform (e.g., having a mean equal to zero and a standard deviation equal to one). In other embodiments, statistical transforms other than Z-score statistical transforms may be used to generate the set of transformed petrophysical properties at block 18. For example, in some embodiments, Poisson regression or other statistical description may be used. The choice of transformation used may be dependent on the type of data available in the material library . . . .

In some embodiments, in addition to generating a set of transformed petrophysical properties, block 18 also includes generating an error or score metric based on the set of transformed petrophysical properties. For instance, the petrophysical properties of a subject porous media sample may be similarly transformed based on the statistical transform and then compared against the corresponding set of transformed petrophysical properties obtained from the material library. By comparing the transformed petrophysical properties of the subject porous media sample against the set of transformed petrophysical properties obtained from the material library, one or more analog materials can be identified from the set of reference porous media samples obtained from the material library in order of similarity based on minimization of an error or score metric.

As an example, in some embodiments, the score metric determined at block 18 comprises the Euclidean or L2 norm over the vector of scalar transformed petrophysical properties. In other words, block 18 may include determining a Euclidian distance between statistically transformed petrophysical properties of a subject porous media sample and each of a set of similarly statistically transformed petrophysical properties sampled or otherwise obtained from a material library of porous media samples. Alternatively, score metrics other than Euclidian distance may be used to compare the similarity between the petrophysical properties of the subject porous media sample from the set of petrophysical properties obtained from the material library. For instance, in some embodiments, the L1 norm may be used for the similarity metric for other data types to capture changes in residuals that may not be easily discernable when using the L2 norm.

In some embodiments, selectable or adjustable weights may be applied to the different statistically transformed petrophysical properties in determining the score metric depending on the requirements of the given application. For instance, these weights may be user-adjusted to focus or tune the determination of the score metric at block 18 on particular petrophysical properties of interest (e.g., permeability, porosity, formation factor, elastic moduli, and the like). In this manner, by permitting the weights used in determining the score metric to be adjusted, specific attributes or particular types of petrophysical properties may be selectably weighted higher or lower, allowing material analogs based on variable weighting to be identified. Thus, the ordering of material analogs identified from comparing the statistically transformed petrophysical properties of the subject porous media sample and the set of statistically transformed petrophysical properties obtained from the material library may vary depending on the particular weighting applied to the different petrophysical properties by the user.

In some embodiments, at block 18 of method 10, each petrophysical property may be represented by the attribute value ᵢAₘ (where m ranges from 1 to M and i is in the set Sₖ) which is remapped or converted to its statistical z-transform value (or any other statistical transformation) “ᵢA^{z}mᵢ” and a similarity score “score_{r,j}” for a subject sample r relative to samples j in the set Sₖ is computed. Additionally, a set of a priori weights “wₘ” may be incorporated (where m ranges from 1 to M) to allow an attribute importance ranking to be captured in the comparison and thereby reflect the relative importance of each attribute in determining overall similarity. Not intending to be bound by any particular theory, an exemplary comparison is illustrated in Equation (1) below:

$${score}_{r , j} = {\sum }_{m = 1}^{M} ‖ {}_{j} {A}_{m}^{Z} - {}_{r} {A}_{m}^{Z} ‖ {w}_{m} \qquad ( 1 )$$

In some embodiments the similarity metric score_{r,x} may be ranked in increasing order to determine sample similarity for all samples j in the set Sₖ. This ordered similarity metric “^{ro}Scoreᵣ” defines the most likely analog samples to subject sample r, which can be used in additional review or analysis.

At block 20, method 10 includes identifying one or more of the plurality of reference porous media samples as a material analog of the subject porous media sample in response to the one or more identified reference porous media sample meeting a predefined similarity score threshold. For example, a user may establish that for a reference sample to be considered a material analog, it must have a predefined similarity score threshold in certain petrophysical properties (e.g., permeability, grain size, porosity) when compared to the subject porous media sample. In this instance, any reference porous media sample meeting or exceeding the predefined threshold (e.g., a predefined minimum similarity metric such as score_{r,x} shown in Equation (1)) is identified as an analog material which can be used in additional review or analysis, and any sample falling below the predefined similarity score threshold may be excluded.

In some embodiments, blocks 18 and 20 of method 10 include comparing, ranking and identifying a reference porous media sample as a material analog of a subject porous media sample based on the petrophysical properties of the reference porous media samples. For instance, the plurality of reference porous media samples in a material library (e.g., material library 120 of FIG. 5) may be divided into distinct and mutually exclusive groups. Additionally, the digitally estimated petrophysical properties of the subject porous media sample is compared with the digitally estimated petrophysical properties of each of the plurality of reference porous media samples in each group. In this example, the plurality of reference porous media samples in the material library are divided into a first group of reference porous media samples comprising porous media samples that are geographically close to the subject porous media sample, and a second group of reference porous media samples comprising all other reference porous media samples stored in the material library that are not part of the first group of porous media samples. The plurality of reference porous media samples in the first group and the second group are referred herein as “local samples” and “world samples,” respectively. The entire plurality of reference porous media samples stored in the material library are also referred herein as “complete samples.” Only six (6) petrophysical properties (indicated as A, B, C, D, E, and F in Tables 1,2 and 3) are used in this exemplary analysis. Alternatively, greater or less than six petrophysical properties may be used in other analysis.

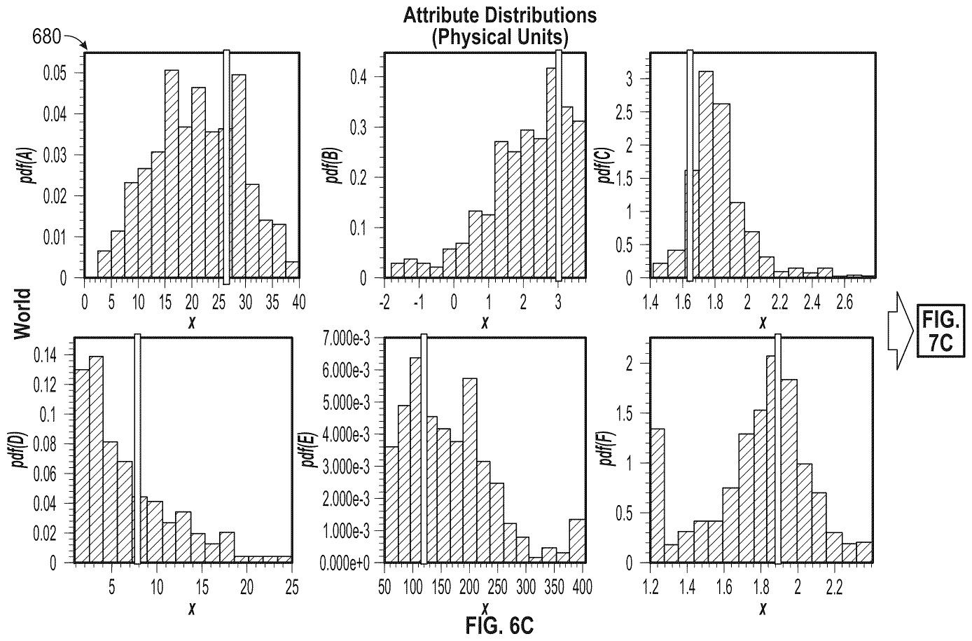

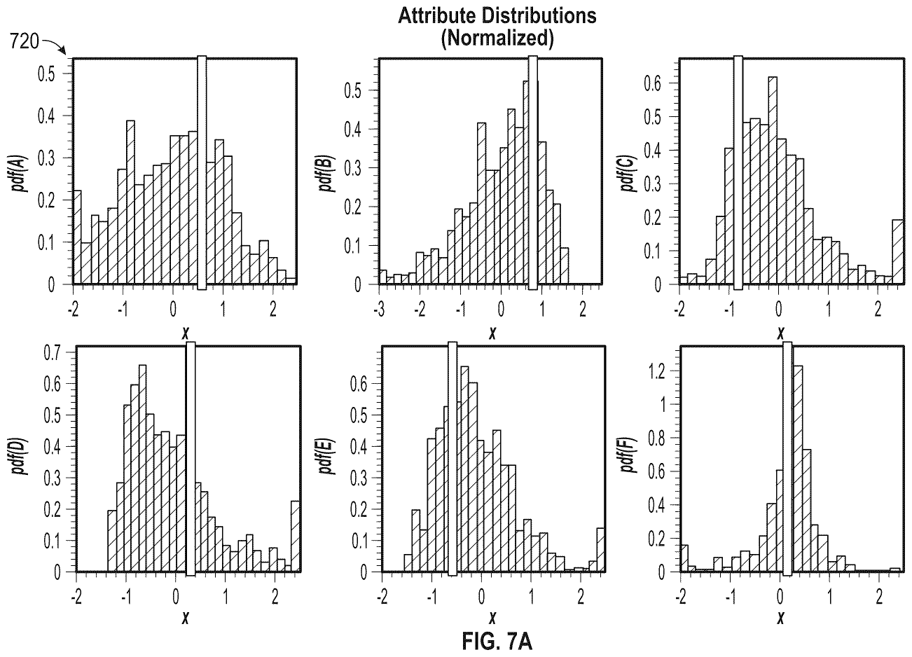

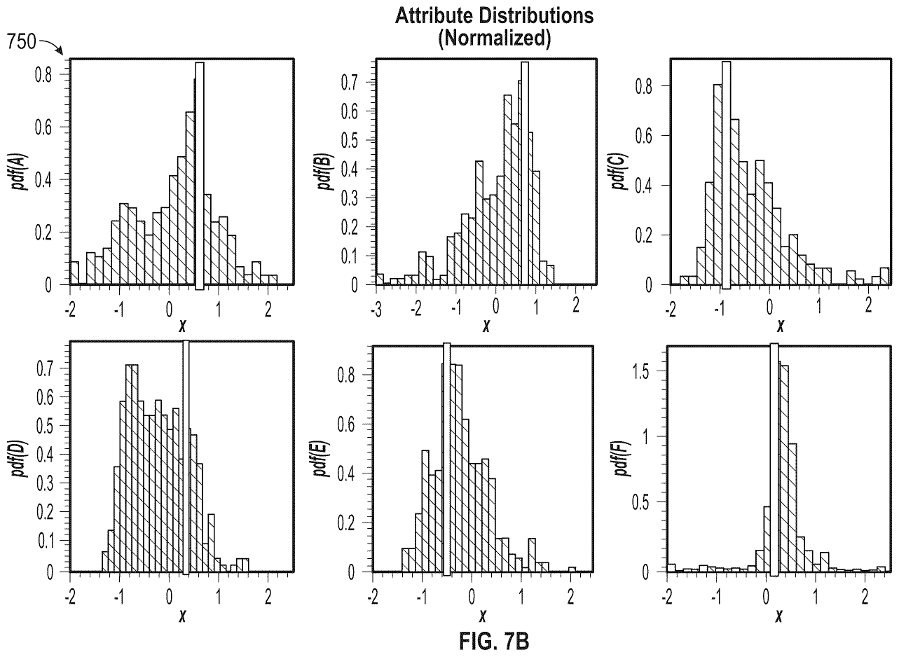

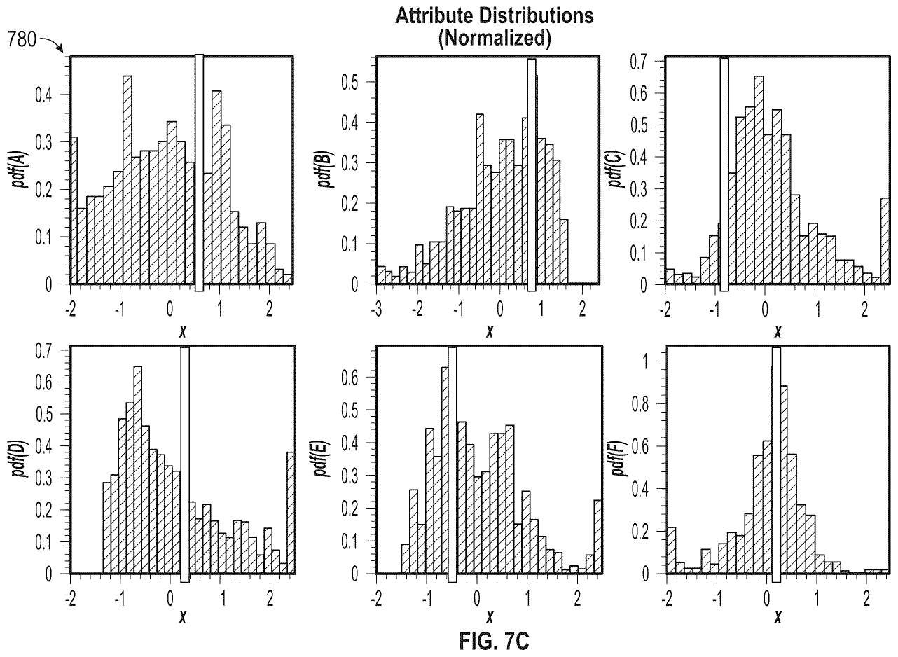

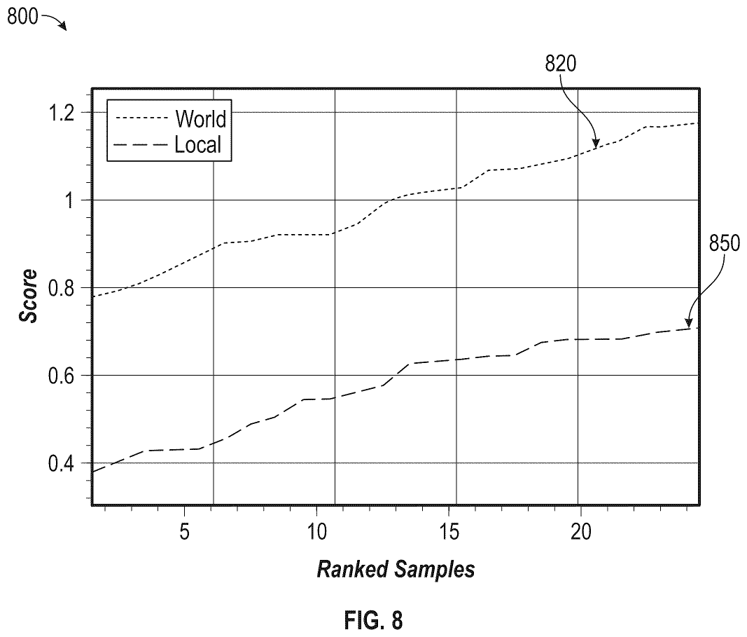

Referring briefly to FIGS. 6-8, bar charts 620, 650, and 680 are included in FIG. 6 that show the petrophysical properties distributions in physical units of the world samples, local samples, and complete samples, respectively. Additionally, FIG. 7 includes bar charts 720, 750, and 780 that show the normalized distributions of the petrophysical properties of the world samples, local samples, and complete samples, respectively. Further, FIG. 8 includes a graph 800 plotting the similarity score (dimensionless) on the Y-axis against the petrophysical properties (in physical units) of the ranked samples on the X-axis. Particularly, graph 800 illustrates a first similarity score 820 of world samples and a second similarity score 850 for local samples for the first 25 ranked samples of the world samples and local samples, respectively, based on equation 1 above.

Particularly, the first four (4) analogs identified from the world samples and local samples along with their corresponding petrophysical properties in physical units are shown in Tables 1 and 2, respectively, below.

TABLE 1

| Sample # | A | B | C | D | E | F |
|---|---|---|---|---|---|---|
| 1 | 29.35 | 3.04 | 1.69 | 7.71 | 107.34 | 1.90 |
| 2 | 29.09 | 2.98 | 1.71 | 7.58 | 104.64 | 1.84 |
| 3 | 24.53 | 2.68 | 1.70 | 5.80 | 115.40 | 1.89 |
| 4 | 24.88 | 2.87 | 1.69 | 6.31 | 137.51 | 2.04 |

TABLE 2

| Sample # | A | B | C | D | E | F |
|---|---|---|---|---|---|---|
| 1 | 24.55 | 2.96 | 1.58 | 7.13 | 128.52 | 1.93 |
| 2 | 26.21 | 2.96 | 1.60 | 6.46 | 129.08 | 1.94 |
| 3 | 25.41 | 3.02 | 1.62 | 7.15 | 137.68 | 1.97 |
| 4 | 27.89 | 3.12 | 1.60 | 7.75 | 122.78 | 1.83 |

Additionally, Table 3 below shows the petrophysical properties of the subject porous media sample in physical units.

TABLE 3

| Sample # | A | B | C | D | E | F |
|---|---|---|---|---|---|---|
| 1 | 26.00 | 3.01 | 1.62 | 7.39 | 119.11 | 1.88 |

As shown in Tables 1 and 2 as compared with Table 3, this example demonstrates the effectiveness of the analog selection method described herein. Based on the analog, a user may be able to predict or estimate physical properties of the subsurface region.

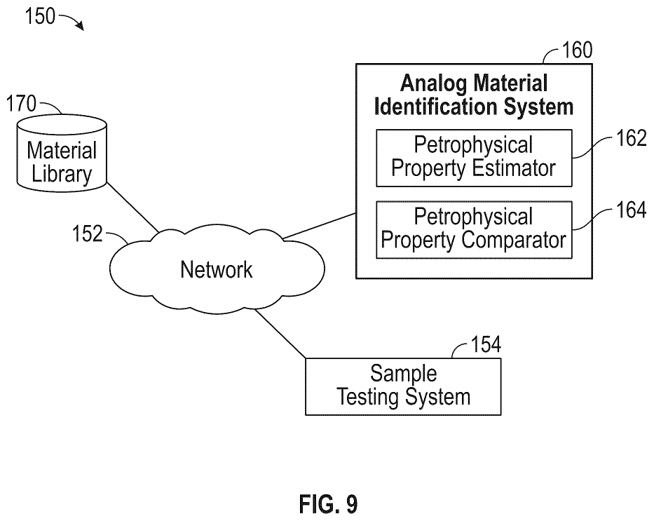

Referring now to FIG. 9, an embodiment of a computer-implemented system 150 is shown. As will be described further herein, system 150 may be utilized to implement method 10 shown in FIG. 1 and/or methods having features in common with method 10 for automatically identifying analog materials based on estimated petrophysical properties of a selected sample material of porous media samples. In this exemplary embodiment, system 150 comprises a material library 170, an analog material identification system 160, a sample testing system 154 and a network 152 providing signal connectivity (e.g., wireless signal connectivity across the Internet) between the sample testing system 154, the analog material identification system 160, and material library 170. The material library 170 may include one or more data stores or digital databases comprising a plurality of distinct materials, each representing a type of porous media or core sample from a subterranean formation and associated with a corresponding plurality of petrophysical properties as previously described and similar to material library 120 shown in FIG. 5. The material library 170 may be communicatively coupled to the analog material identification system 160 via network 152 using a wired or wireless communication link into network 152. The network 152 may be one or more private networks, one or more public networks, or a combination thereof.

Referring briefly to FIG. 10 a flowchart of an exemplary method 180 for adding petrophysical properties to a digital material library (e.g., material library 170 of FIG. 10) is shown. Method 180 begins at block 182 with receiving a plurality of porous media or core samples, and well data from various subterranean formations and reservoirs. The well data may include well logs, seismic data, and other data for identifying porous media samples and/or correlating data from the various subterranean formations and reservoirs. At block 184, method 180 continues with selecting a set of porous media samples from the plurality of porous media samples received at block 182. For example, k number of porous media samples may be selected from N porous media samples received, based on the quality or specific attribute or property of each of the N porous media samples. For instance, samples having uniform texture, consistent grain size, stable physical integrity and so on may be selected while samples with visible anomalies, cracks or fractures, compromised integrity, and so on, may be excluded. Method 180 continues at block 186 with generating a digital image volume of each of the k selected porous media samples. In some embodiments, the generated digital image volume is a three-dimensional (3D) representation of the porous media sample generated using micro-computed tomography (Micro-CT).

At block 188, method 180 continues with determining petrophysical properties of each of the porous media samples in the selected set. In some embodiments, the petrophysical properties may be digitally estimated or measured based direct numerical simulation or other numerical or synthetic methods to determine the petrophysical properties associated with each porous media sample in the selected set. In some instances, relationships between measured variables and petrophysical properties may be estimated based on statistical methods (e.g., least-square maps) to identify trends, evaluate existing models, or to provide indicators requiring further investigation.

Method 180 continues at block 190 with providing or adding the petrophysical properties of the selected porous media samples to a digital material library. The petrophysical properties of the porous media samples may be provided or added to a digital material library for storage along with a corresponding identifier of each porous media sample. In some embodiments, the petrophysical properties may be indexed in the material library with a corresponding label or identifier for each of the k selected samples. The identifier may be based on the type of material of the porous media sample, the location at which the sample was obtained, or other identifying characteristics. In this manner, the material library provides access to already tested and validated samples that may be used for comparison with newly acquired subject porous media samples.

Referring back to FIG. 9, analog material identification system 160 is communicatively coupled to sample testing system 154 via network 152, and includes a petrophysical property estimator 162 and petrophysical property comparator 164. The analog material identification system 160 may be a computer system, software/hardware, with one or more processors, memories, and/or network resources used to implement one or more applications. For example, a set Sₖ of samples may be received from material library 170 by petrophysical property comparator 164 to thereby determine a similarity score between a petrophysical property of a one or more reference sample in the set Sₖ and a corresponding petrophysical property of a subject porous media sample. In this manner, the petrophysical property comparator 164 serves to analyze and contrast the petrophysical properties of various porous media types to identify analogs that exhibit comparable characteristics.

In this embodiment, the subject porous media sample is provided by sample testing system 154. The sample testing system 154 may analyze samples of porous media in the form of, for example, porous media samples obtained from a subsurface region and provide measurements deduced thereof to petrophysical property estimator 162. The subject porous media samples may be digitally imaged and analyzed by sample testing system 154. The processes executed by sample testing system 154 may be like the processes executed by testing system 52 of FIG. 3 as previously described. For example, sample testing system 154 may provide saturation measurements, pore size and fluid distribution, young's modulus and Poisson's ratio, temperature and pressure measurements, etc., to the petrophysical property estimator 162 from which petrophysical properties of the subject sample is estimated. As previously described, the petrophysical properties estimated by petrophysical property estimator 162 may include, among other parameters, estimates of porosity, permeability and other relevant properties.

While FIG. 9 only includes material library 170, analog material identification system 160, sample testing system 154, and network 152, it should be appreciated that system 150 may include a plurality of material libraries or other components not shown in FIG. 9. The process of retrieving petrophysical property data associated with reference porous media samples from material library 170 allows for access to already tested and validated samples for comparison with newly received subject porous media samples. Comparison of the petrophysical properties of subject samples with the petrophysical properties of reference samples from material library 170, allows for faster, more efficient and reliable selection of analog samples, thereby enhancing the accuracy of petrophysical analysis.

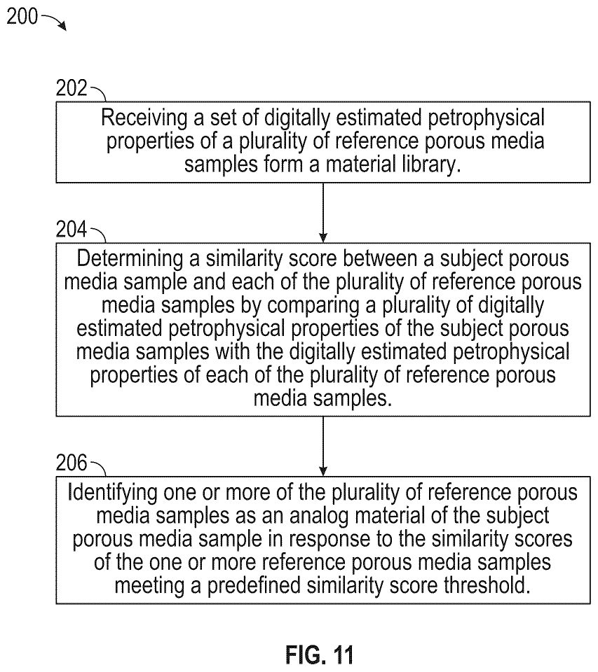

Referring to FIG. 11, an embodiment of a computer-implemented method 200 for automatically identifying analog materials of a subject porous media sample is shown. It may be understood that identifying analog materials of a subject porous media sample based on digitally estimated petrophysical properties of the subject porous media sample described by method 200 may be used for a variety of purposes, including volumetric analysis and in the planning of one or more wells which would extend through a subsurface region. Particularly, and as further discussed below, method 200 may incorporate at least some of the features or steps of method 10 described above and shown in FIG. 1.

Method 200 begins at block 202 with receiving a set of digitally estimated petrophysical properties of a plurality of reference porous media samples from a material library. As previously described the material library may comprise petrophysical properties of reference porous media samples digitally estimated by a sample testing system and provided or added to the material library for storage therein. Particularly, the petrophysical properties in the material library may be indexed with a corresponding label or identifier of the given porous media sample. The identifier of the reference porous media sample may be based on the type of material forming the reference porous media sample, the location at which the reference porous media was sampled, or other identifying characteristics. In other embodiments, the identifier of the reference porous media sample may be arbitrarily or randomly assigned.

At block 204, method 200 continues with determining a similarity score between a subject porous media sample and each of the plurality of reference porous media samples by comparing a plurality of digitally estimated petrophysical properties of the subject porous media samples with the digitally estimated petrophysical properties of each of the plurality of reference porous media samples. As previously described, determining a similarity score between the subject porous media sample and each of the plurality of reference porous media samples include leveraging numerical (e.g., statistical) methods to quantitatively determine the similarity between petrophysical properties of the subject porous media sample and the petrophysical properties of each of the reference porous media samples received from the material library. In some embodiments, an attribute associated with a subject porous media sample may be tested against the equivalent attribute of reference porous media samples stored in the material library based on minimization of an error metric. In this embodiment, the digitally estimated petrophysical properties associated with a subject porous media sample is tested against the equivalent petrophysical properties of reference samples stored in the material library.

At block 206, method 200 continues with identifying one or more of the plurality of reference porous media samples as an analog material of the subject porous media sample in response to the similarity score of the one or more reference porous media samples meeting a predefined similarity score threshold. As used herein, “predefined similarity score threshold” refers to a particular similarity score or the most similar to the subject porous media sample among the set of reference porous media samples. For example, a user may establish that for a reference sample to be considered an analog, it must have a predefined similarity score threshold in certain petrophysical properties (e.g., permeability, grain size, porosity) when compared to the subject porous media sample. In this instance, any reference porous media sample meeting or exceeding the redefined similarity score threshold is selected as an analog material of the subject porous media sample, which can be used in additional review or analysis, and any reference porous media sample falling below the predefined similarity score threshold may be excluded.

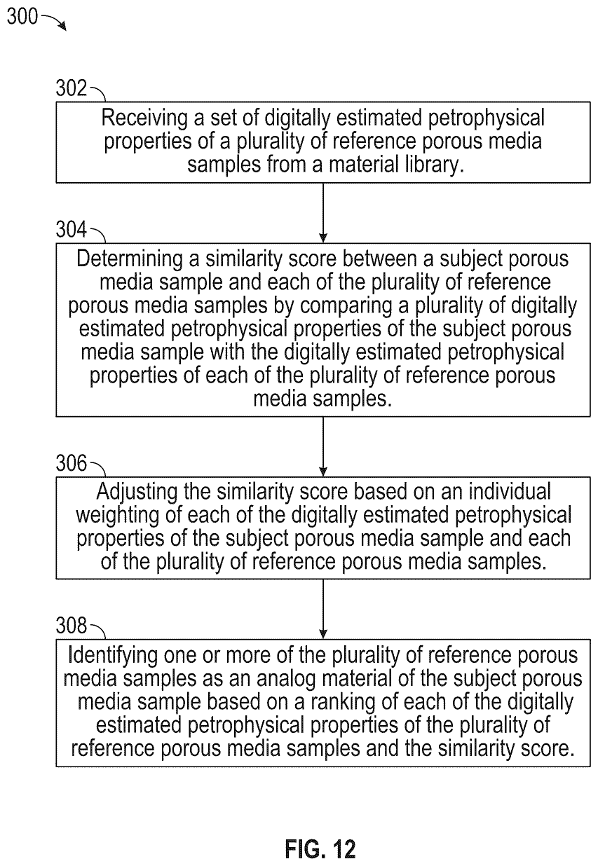

Referring to FIG. 12, an embodiment of another computer-implemented method 300 for automatically identifying analog materials of a subject porous media sample is shown. It may be understood that identifying analog materials of a subject porous media sample based on digitally estimated petrophysical properties of the subject porous media sample described by method 300 may be used for a variety of purposes, including volumetric analysis and in the planning of one or more wells which would extend through a subsurface region. Particularly, and as further discussed below, method 300 may incorporate at least some of the features or steps of method 10 and/or method 200 described above and shown in FIGS. 1 and 11 respectively.

Method 300 begins at block 302 with receiving a set of digitally estimated petrophysical properties of a plurality of reference porous media samples from a material library. As previously described the material library may comprise petrophysical properties of reference porous media samples digitally estimated by a sample testing system (e.g., sample testing system 154 of FIG. 150) and provided or added to the material library for storage therein. Particularly, the petrophysical properties in the material library may be indexed with a corresponding label or identifier of the given porous media sample. The identifier of the reference porous media sample may be based on the type of material forming the reference porous media sample, the location at which the reference porous media was sampled, or other identifying characteristics. In other embodiments, the identifier of the reference porous media sample may be arbitrarily or randomly assigned.

At block 304, method 300 continues with determining a similarity score between a subject porous media sample and each of the plurality of reference porous media samples by comparing a plurality of digitally estimated petrophysical properties of the subject porous media samples with the digitally estimated petrophysical properties of each of the plurality of reference porous media samples. As previously described, determining a similarity score between the subject porous media sample and each of the plurality of reference porous media samples include based on numerical methods to quantitatively determine similarity scores between the petrophysical properties of the subject porous media sample and the petrophysical properties of each of the reference porous media samples received from the material library. In some embodiments, the petrophysical properties associated with a subject porous media sample may be tested against the equivalent petrophysical properties of each of the reference porous media samples stored in the material library based on minimization of an error metric.

Method 300 continues at block 306 with adjusting the similarity score based on an individual weighting of each of the digitally estimated petrophysical properties of the subject porous media sample and each of the plurality of reference porous media samples. In some embodiments, user-selectable weights may be assigned to each digitally estimated petrophysical property and incorporated in the similarity score to allow an attribute importance ranking to be captured in the comparison. For example, if porosity is assigned a weight of 0.3 out of 1 and saturation is assigned a weight of 0.2 out of 1, porosity would contribute 30% of the overall similarity score, while saturation will contribute 20%. In this manner certain petrophysical properties are prioritized over others, such that the resulting similarity scores become a more accurate and meaningful representation of the relationship between the subject porous media samples and the reference samples based on the relative importance of each petrophysical property within the framework of the similarity analysis.

At block 308, method 300 continues with identifying one or more of the plurality of reference porous media samples as an analog material of the subject porous media sample based on a ranking of each of the digitally estimated petrophysical properties of the plurality of reference porous media samples and the similarity score. In some embodiments, each of the plurality of reference porous media samples may be ranked based on the digitally estimated petrophysical properties and overall similarity score which includes the user-selectable weight of each of the digitally estimated petrophysical property (e.g., in decreasing order of similarity score). In this manner, analogs may be selected based on the relative importance of a digitally estimated petrophysical property and/or similarity to the subject porous media sample.

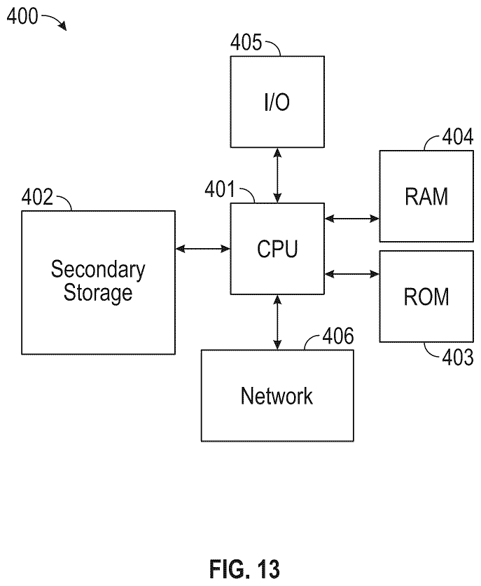

Any of the systems and methods disclosed herein can be carried out (e.g., entirely or partially) on a computer or other device comprising a processor (e.g., a desktop computer, a laptop computer, a tablet, a server, a smartphone, or some combination thereof). Referring now to FIG. 13, a computer system 400 suitable for implementing one or more embodiments disclosed herein is shown. The computer system 400 includes a processor 401 (which may be referred to as a central processor unit or CPU) that is in communication with memory devices including secondary storage 402, read only memory (ROM) 403, random access memory (RAM) 404, input/output (I/O) devices 405, and network connectivity devices 406. The processor 401 may be implemented as one or more CPU chips.

It is understood that by programming and/or loading executable instructions onto the computer system 400, at least one of the CPUs 401, the RAM 404, and the ROM 403 are changed, transforming the computer system 400 in part into a particular machine or apparatus having the novel functionality taught by the present disclosure. Thus, the RAM 404 and/or the ROM 403 may comprise a non-transitory machine-readable (or computer-readable) medium that may include instructions (which may be referred to herein as machine-readable instructions) that are executable by CPU 401 to provide functionality to computer system 400. Thus, in some embodiments, a machine-readable instructions stored on a memory may be executed on a processor, so as to configure the processor to carry out some or all of the features of the methods described herein (e.g., method 10 shown in FIG. 1).

It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware (for example in an application specific integrated circuit (ASIC), or field-programmable gate arrays (FPGA)) because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the system 400 is turned on or booted, the CPU 401 may execute a computer program or application. For example, the CPU 401 may execute software or firmware stored in the ROM 403 or stored in the RAM 404. In some cases, on boot and/or when the application is initiated, the CPU 401 may copy the application or portions of the application from the secondary storage 402 to the RAM 404 or to memory space within the CPU 401 itself, and the CPU 401 may then execute instructions of which the application is comprised. In some cases, the CPU 401 may copy the application or portions of the application from memory accessed via the network connectivity devices 406 or via the I/O devices 405 to the RAM 404 or to memory space within the CPU 401, and the CPU 401 may then execute instructions of which the application is comprised. During execution, an application may load instructions into the CPU 401, for example load some of the instructions of the application into a cache of the CPU 401. In some contexts, an application that is executed may be said to configure the CPU 401 to do something, e.g., to configure the CPU 401 to perform the function or functions promoted by the subject application. When the CPU 401 is configured in this way by the application, the CPU 401 becomes a specific purpose computer or a specific purpose machine.

The secondary storage 402 is typically comprised of one or more disk drives or tape drives and is used for non-volatile storage of data and as an over-flow data storage device if RAM 404 is not large enough to hold all working data. Secondary storage 402 may be used to store programs which are loaded into RAM 404 when such programs are selected for execution. The ROM 403 is used to store instructions and perhaps data which are read during program execution. ROM 403 is a non-volatile memory device which typically has a small memory capacity relative to the larger memory capacity of secondary storage 402. The RAM 404 is used to store volatile data and perhaps to store instructions. Access to both ROM 403 and RAM 404 is typically faster than to secondary storage 402. The secondary storage 402, the RAM 404, and/or the ROM 403 may be referred to in some contexts as computer readable storage media and/or non-transitory computer readable media.

I/O devices 405 may include printers, video monitors, electronic displays (e.g., liquid crystal displays (LCDs), plasma displays, organic light emitting diode displays (OLED), touch sensitive displays, etc.), keyboards, keypads, switches, dials, mice, track balls, voice recognizers, card readers, paper tape readers, or other well-known input devices.

The network connectivity devices 406 may take the form of modems, modem banks, Ethernet cards, Omni-Path Architecture (OPA), InfiniBand (IB), universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 406 may enable the processor 401 to communicate with the Internet or one or more intranets. With such a network connection, it is contemplated that the processor 401 might receive information from the network, or might output information to the network (e.g., to an event database) in the course of performing the methods (e.g., method 10 shown in FIG. 1) described herein. Such information, which is often represented as a sequence of instructions to be executed using processor 401, may be received from and outputted to the network, for example, in the form of a computer data signal embodied in a carrier wave.

Such information, which may include data or instructions to be executed using processor 401 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several known methods. The baseband signal and/or signal embedded in the carrier wave may be referred to in some contexts as a transitory signal.

The processor 401 executes instructions, codes, computer programs, scripts which it accesses from hard disk, floppy disk, optical disk, solid state drives (SSD) (these various disk-based systems may all be considered secondary storage 402), flash drive, ROM 403, RAM 404, or the network connectivity devices 406. While only one processor 401 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 402, for example, hard drives, floppy disks, optical disks, and/or other device, the ROM 403, and/or the RAM 404 may be referred to in some contexts as non-transitory instructions and/or non-transitory information.

In an embodiment, the computer system 400 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 400 to provide the functionality of a number of servers that is not directly bound to the number of computers in the computer system 400. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid-state memory chip, for example analog magnetic tape, compact disk read only memory (CD-ROM) disks, floppy disks, jump drives, digital cards, multimedia cards, and others. The computer program product may be suitable for loading, by the computer system 400, at least portions of the contents of the computer program product to the secondary storage 402, to the ROM 403, to the RAM 404, and/or to other non-volatile memory and volatile memory of the computer system 400. The processor 401 may process the executable instructions and/or data structures in part by directly accessing the computer program product, for example by reading from a CD-ROM disk inserted into a disk drive peripheral of the computer system 400. Alternatively, the processor 401 may process the executable instructions and/or data structures by remotely accessing the computer program product, for example by downloading the executable instructions and/or data structures from a remote server through the network connectivity devices 406. The computer program product may comprise instructions that promote the loading and/or copying of data, data structures, files, and/or executable instructions to the secondary storage 402, to the ROM 403, to the RAM 404, and/or to other non-volatile memory and volatile memory of the computer system 400.

In some contexts, the secondary storage 402, the ROM 403, and the RAM 404 may be referred to as a non-transitory computer readable medium or a computer readable storage media. A dynamic RAM embodiment of the RAM 404, likewise, may be referred to as a non-transitory computer readable medium in that while the dynamic RAM receives electrical power and is operated in accordance with its design, for example during a period of time during which the computer system 400 is turned on and operational, the dynamic RAM stores information that is written to it. Similarly, the processor 401 may comprise an internal RAM, an internal ROM, a cache memory, and/or other internal non-transitory storage blocks, sections, or components that may be referred to in some contexts as non-transitory computer readable media or computer readable storage media.

The discussion above is directed to various exemplary embodiments. However, one of ordinary skill in the art will understand that the examples disclosed herein have broad application, and that the discussion of any embodiment is meant only to be exemplary of that embodiment and not intended to suggest that the scope of the disclosure, including the claims, is limited to that embodiment.

The drawing figures are not necessarily to scale. Certain features and components herein may be shown exaggerated in scale or in somewhat schematic form and some details of conventional elements may not be shown in interest of clarity and conciseness.

In the discussion above and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, when used herein (including in the claims), the words “about,” “generally,” “substantially,” “approximately,” and the like mean within a range of plus or minus 10%.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps but rather are used to simplify subsequent reference to such steps.

What is claimed is:

1. A computer implemented method for automatically identifying analog materials of a subject porous media sample, the method comprising:
(a) receiving a set of digitally estimated petrophysical properties of a plurality of reference porous media samples from a material library;
(b) determining a similarity score between a subject porous media sample and each of the plurality of reference porous media samples by comparing a plurality of digitally estimated petrophysical properties of the subject porous media sample with the digitally estimated petrophysical properties of each of the plurality of reference porous media samples; and
(c) identifying one or more of the plurality of reference porous media samples as an analog material of the subject porous media sample in response to the similarity scores of the one or more reference porous media samples meeting a predefined similarity score threshold.

2. The method of claim 1, wherein (b) comprises:
(b1) statistically transforming each of the digitally estimated petrophysical properties of the plurality of reference porous media samples to generate a corresponding plurality of transformed petrophysical properties of the plurality of reference porous media samples;
(b2) statistically transforming each of the digitally estimates petrophysical properties of the subject porous media sample to generate a corresponding plurality of transformed petrophysical properties of the reference porous media sample; and
(b3) determining the similarity score by comparing the plurality of transformed petrophysical properties of the subject porous media sample with the plurality of transformed petrophysical properties of each of the plurality of reference porous media samples.

3. The method of claim 2, wherein:
(b1) comprises applying a Z-score statistical transformation to each of the digitally estimated petrophysical properties of the plurality of reference porous media samples; and
(b2) comprises applying a Z-score statistical transformation to each of the digitally estimates petrophysical properties of the subject porous media sample.

4. The method of claim 2, wherein the similarity score comprises a set of Euclidean distances between the plurality of transformed petrophysical properties of the subject porous media sample and the plurality of transformed petrophysical properties of each of the plurality of reference porous media samples.

5. The method of claim 2, wherein (b3) comprises individually weighting one or more of the plurality of transformed petrophysical properties of the subject porous media sample and the plurality of transformed petrophysical properties of each of the plurality of reference porous media samples.

6. The method of claim 5, wherein (b) comprises defining a set of user-selectable weights for each of the digitally estimated petrophysical properties of the plurality of reference porous media samples.

7. The method of claim 6, wherein (b) further comprises incorporating the set of the user-selectable weights in the similarity score.

8. The method of claim 1, wherein (b) comprises ranking the plurality of reference porous media samples in order of decreasing similarity score.

9. A computer implemented method for automatically identifying analog materials of a subject porous media sample, the method comprising:
(a) receiving a set of digitally estimated petrophysical properties of a plurality of reference porous media samples from a material library;
(b) determining a similarity score between a subject porous media sample and each of the plurality of reference porous media samples by comparing a plurality of digitally estimated petrophysical properties of the subject porous media sample with the digitally estimated petrophysical properties of each of the plurality of reference porous media samples;
(c) adjusting the similarity score based on an individual weighting of each of the digitally estimated petrophysical properties of the subject porous media sample and each of the plurality of reference porous media samples; and
(d) identifying one or more of the plurality of reference porous media samples as an analog material of the subject porous media sample based on a ranking of each of the digitally estimated petrophysical properties of the plurality of reference porous media samples and the similarity score.

10. The method of claim 9, wherein (b) comprises:
(b1) statistically transforming each of the digitally estimated petrophysical properties of the plurality of reference porous media samples to generate a corresponding plurality of transformed petrophysical properties of the plurality of reference porous media samples;
(b2) statistically transforming each of the digitally estimates petrophysical properties of the subject porous media sample to generate a corresponding plurality of transformed petrophysical properties of the reference porous media sample; and
(b3) determining the similarity score by comparing the plurality of transformed petrophysical properties of the subject porous media sample with the plurality of transformed petrophysical properties of each of the plurality of reference porous media samples.

11. The method of claim 10, wherein:
(b1) comprises applying a Z-score statistical transformation to each of the digitally estimated petrophysical properties of the plurality of reference porous media samples.

12. The method of claim 11, wherein:
(b2) comprises applying a Z-score statistical transformation to each of the digitally estimates petrophysical properties of the subject porous media sample.

13. The method of claim 9, wherein (c) comprises individually weighting the digitally estimated petrophysical properties making up the plurality of digitally estimated petrophysical properties of the subject porous media sample and the plurality of digitally estimated petrophysical properties of each of the plurality of reference porous media samples.

14. The method of claim 13, wherein (c) further comprises defining a set of user-selectable weights for each of the digitally estimated petrophysical properties of the plurality of reference porous media samples.

15. The method of claim 14, wherein (c) further comprises incorporating the set of the user-selectable weights in the similarity score.

16. The method of claim 9, wherein (d) comprises ranking the plurality of reference porous media samples in order of decreasing similarity score.

17. A system for automatically identifying analog materials of a subject porous media sample, the system comprising:
a one or more processors; and
a storage device coupled to the one or more processors, the storage device configured to store instructions that, when executed by the one or more processors, configure the one or more processors to:
(a) receive a set of digitally estimated petrophysical properties of a plurality of reference porous media samples from a material library;
(b) determine a similarity score between a subject porous media sample and each of the plurality of reference porous media samples by comparing a plurality of digitally estimated petrophysical properties of the subject porous media sample with the digitally estimated petrophysical properties of each of the plurality of reference porous media samples; and
(c) identify one or more of the plurality of reference porous media samples as an analog material of the subject porous media sample in response to the similarity scores of the plurality of reference porous media samples meeting a predefined similarity score threshold.

18. The system of claim 17, wherein the storage device is configured to store instructions that, when executed by the one or more processors, further configure the one or more processors to:
(b1) statistically transform each of the digitally estimated petrophysical properties of the plurality of reference porous media samples to generate a corresponding plurality of transformed petrophysical properties of the plurality of reference porous media samples;
(b2) statistically transform each of the digitally estimates petrophysical properties of the subject porous media sample to generate a corresponding plurality of transformed petrophysical properties of the reference porous media sample; and
(b3) determine the similarity score by comparing the plurality of transformed petrophysical properties of the subject porous media sample with the plurality of transformed petrophysical properties of each of the plurality of reference porous media samples.

19. The system of claim 18, wherein the storage device is configured to store instructions that, when executed by the one or more processors, further configure the one or more processors to:
(b1) apply a Z-score statistical transformation to each of the digitally estimated petrophysical properties of the plurality of reference porous media samples; and
(b2) apply a Z-score statistical transformation to each of the estimated petrophysical properties of the subject porous media sample.

20. The system of claim 18, wherein the storage device is configured to store instructions that, when executed by the one or more processors, further configure the one or more processors to:
(b3) individually weight one or more of the transformed petrophysical properties of the subject porous media sample and the plurality of transformed petrophysical properties of each of the plurality of reference porous media samples.
